(19) United States
(12) Patent Application Publication
(10) Pub. No.: US 2012/0301538 A1
(43) Pub. Date: Nov. 29, 2012

(54) PHARMACEUTICAL COMPOSITIONS FOR THE STIMULATION OF STEM CELLS

(76) Inventors: Roland GORDON-BERESFORD, La Hulpe (BE); Vinciane Gaussin, Wezembeek-oppem (BE); Jean-Pierre Latere Dwan'isa, Namur (BE); Christian Homsy, Bruxelles (BE)

(21) Appl. No.: 13/484,956

(22) Filed: May 31, 2012

Related U.S. Application Data
(63) Continuation of application No. PCT/EP2010/068700, filed on Dec. 2, 2010.

(51) Int. Cl. A61K 38/18; A61K 38/20; A61K 38/19; A61K 38/46; A61K 9/14; A61K 9/127; A61K 9/48; A61P 9/00; A61K 38/30; A61K 38/48; B82Y 5/00

(52) U.S. Cl. 424/450; 514/8.6; 514/8.9; 514/8.8; 514/9.1; 514/7.6; 424/85.2; 514/9.6; 514/7.7; 424/85.1; 514/9.5; 514/8.1; 424/94.6; 424/94.65; 424/400; 424/451; 977/788; 977/906

(57) ABSTRACT

The invention relates to a human or veterinary pharmaceutical composition (B) for the stimulation of stem cells, comprising at least two stem-cells-stimulating-agents and at least one pharmaceutically acceptable excipient.

This application is a continuation of PCT/EP2010/068700, filed Dec. 2, 2010; which claims the priority of PCT/EP2009/066251, filed Dec. 2, 2009. The contents of the above-identified applications are incorporated herein by reference in their entirety.

### TECHNICAL FIELD

The present invention relates generally to pharmaceutical compositions suitable for targeting tissues and/or organs. In particular, it relates to the treatment of heart diseases through administration of stem cell-stimulating agents to the heart of an individual in need of heart tissue regeneration. In particular, it discloses the use of such stem cell-stimulating agents to improve the regeneration of cardiac tissue from cardiac stem cells in vivo.

### DESCRIPTION OF RELATED ART

Myocardial infarction (MI) results in loss of cardiomyocytes, scar formation, ventricular remodeling, and eventually heart failure. Pharmacologic, catheter-based, and surgical interventions have led to improved survival of patients with coronary artery disease (CAD), although they fail to regenerate dead myocardium. Consequently, reduced mortality is accompanied by increased morbidity because of ischemic heart failure. In recent years, stem cell-based therapy has emerged as a potential new strategy for cardiac repair (Dimmeler S. et al., J Clin Invest 2005, 11, 572-583). The optimal source of cells for repairing damaged myocardium is a topic of intense research. Important features of stem cells for cardiac regeneration include self renewal, clonogenicity, and the ability to differentiate into cardiomyocytes, endothelial cells and vascular smooth muscle cells.

Over the past 10 years, researchers have applied various bone marrow (BM)-derived stem/progenitor cells for cardiac reparative therapy in animal studies, such as lineage negative (lin neg) c-kit positive (c-kit pos) BM stem cells, (Orlic et al., Nature 2001; 410: 701-705; Kajstura et al., Circ. Res. 2005; 96: 127-137; Rota et al., Proc Natl Acad Sci USA 2007; 104: 17783-17788) BM-derived mesenchymal stem cells (MSCs) (Min et al., Ann Thor Surg 2002; 74: 1568-1575; Amado et al., Proc Natl Acad Sci USA 2005; 102: 11474-11479) and endothelial progenitor cells (EPCs) (Cho et al., J Exp Med 2007; 204: 3257-3269; Schuh et al., Basic Res Cardiol 2008; 103:60-77). Despite these studies showing the differentiation of BM-derived stem/progenitor cells into cells with hallmark features of cardiomyocytes and vascular cells, other studies suggested that the transplanted BM stem cells do not readily acquire a cardiac phenotype in the injured heart (Balsam et al., Nature 2004; 428: 668-673; Murry et al., Nature 2004; 428: 664-668; Nygren et al., Nat Med 2004; 10: 494-501).

Thus, enhancing differentiation of BM-derived stem/progenitor cells after their transplantation remains a great challenge for researchers to effectively use these cells in cardiac regenerative therapy. Other stem cell sources may be used for cardiac regenerative therapy apart from BM-derived stem/progenitor cells. In particular, resident cardiac stem cells (CSCs) were discovered in the heart itself (Beltrami et al. Cell 2003; 114: 763-776; Uranek et al., Proc Natl Sci USA 2006; 103: 9226-9231). Because CSCs already reside within the heart and are programmed to generate cardiac tissue, they represent a logical source to exploit in cardiac regenerative therapy, when massive loss of cardiac tissue occurred. Given that CSCs have unique characteristics, the identification of resident CSCs created great excitement and sparked intense hope for myocardial regeneration with cells that are from the heart itself and are thereby inherently programmed to reconstitute cardiac tissue.

Myocardial repair requires the formation of new myocytes and coronary vessels, and it cannot be accomplished by a cell already fully committed to the myocyte lineage. In the presence of an infarct, the generation of myocytes alone cannot restore contractile performance in the akinetic region; myocytes will not grow or survive in the absence of vessel formation. Arterioles are critical for blood supply, and oxygen delivery is controlled by the capillary network. Similarly, neovasculogenesis alone would not restore the dead myocardium or reinstitute contractile activity in the infarcted portion of the ventricular wall. Observation that CSCs injected locally in the infarcted myocardium of animals repaired the necrotic tissue and improve ventricular function (Beltrami et al. Cell 2003; 114: 763-776; Bearzi et al., Proc Natl Sci USA 2007; 104: 14068-14073) has formed the basis of a new paradigm in which CSCs are implicated in the normal renewal of myocytes, endothelial cells, smooth muscle cells, and fibroblasts. In an attempt to develop strategies relevant to the future treatment of patients, new hypotheses have to be raised to move the field in a direction that defines CSCs therapy clinically on an individual basis.

Various attempts have thus been made to deal with the discovered CSCs for clinical applications. The first approach is isolation, culture, cloning and expansion of CSCs. Such cells would be injected back into the infracted heart in an attempt to regenerate functional myocardium. However, the scarcity of the CSC cell population, combined to stringent cell culture conditions and poor yield, are limiting factors using this approach. The other alternative described in the art is the recruitment and differentiation of endogenous CSCs using exogenous agents. However, no clear evidence on the efficacy of this approach has been described casting uncertainty on the capacity to effectively recruit and differentiate CSCs in vivo.

### SUMMARY OF THE INVENTION

The present invention provides a totally novel approach to stimulate in vivo resident CSCs and, in one aspect, to commit them into the cardiac lineage, particularly to obtain from them a significant number of satisfactorily functional cells with hallmark features of cardiomyocytes.

The invention relates to a human or veterinary pharmaceutical composition (B) for the stimulation of stem cells, comprising at least two stem cells-stimulating-agents and at least one pharmaceutically acceptable excipient.

Said at least two stem cells-stimulating-agents may be selected in the group consisting of TGFβ-1, BMP-4, FGF-2, IGF-1, Activin-A, alpha-thrombin, Cardiotrophin 1, Cardiogenol C and mixtures thereof. In particular, said at least two stem cells-stimulating-agents may be selected in the group consisting of TGFβ-1, BMP-4, FGF-2, IGF-1, Activin-A, Cardiotrophin 1, Cardiogenol C and mixtures thereof.

The invention also relates to a pharmaceutical cocktail comprising a pharmaceutical composition (B) according to the present invention and a composition (A) comprising at least one pharmaceutically active substance. The composition or cocktail of the present invention allows to provide stimulating agent-guided stem cells which means that resident cardiac stem cells, after having been put into contact with the composition or cocktail, are stimulated to enter into differentiation. Hence, the stimulated stem cells may be committed into a cardiac lineage and may become a cardiomyocyte.

Within the frame of the present document, and unless indication of the contrary, the terms designated below between quotes have the following definitions.

As used herein, the term “stimulation or stimulating” refers to recruitment, proliferation, survival and/or differentiation of stem cells.

As used herein, the terms “cardiac tissue” and “myocardium” refer to myocytes, blood vessels and fibroblasts.

‘Cardiac stem cells’ (CSCs), ‘cardiac progenitor cells’, ‘resident cardiac stem cells’ or ‘resident cardiac progenitor cells’ designate stem cells which are present in the myocardium. They are self-renewing, clonogenic, multipotent and may generate myocardium.

A ‘stem cells stimulating agent’ is an agent which improves the ability of stem cells, to be recruited to the site to be regenerated, to proliferate and to differentiate into cardiac tissue.

A ‘stem cells stimulating agent composition’ is a composition comprising at least two stem cell stimulating agents.

A ‘stimulating agent-guided stem cell’ is a stem cell which was in contact with a stem cell stimulating agent composition as defined above and further enters into differentiation i.e. is committed into the cardiac lineage.

The ‘differentiation’ is the process by which a less specialized cell becomes a more specialized cell.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control the meaning of terms of the present invention. In addition, the materials, methods, and examples are illustrative only and not intended to be limiting.

### DETAILED DESCRIPTION OF THE INVENTION

In a preferred embodiment, the pharmaceutical composition (B) may comprise at least five stem cells-stimulating-agents selected in the group consisting of TGFβ-1, BMP-4, FGF-2, IGF-1, Activin-A, Cardiotrophin 1, Cardiogenol C and mixtures thereof.

In particular, the pharmaceutical composition (B) may comprise TGFβ-1, BMP-4, FGF-2, IGF-1, Activin-A, Cardiotrophin 1, and Cardiogenol C.

Moreover, said pharmaceutical composition (B) may further optionally comprise alpha-thrombin. Said pharmaceutical composition (B) may further comprise thrombin inhibitors, such as hirudin, bivalirudin, lepirudin, deirudin, argatroban, melagatran, ximelagatran, dabigatran, and heparin. Alpha-thrombin is a coagulant agent. Alternatively, in some cases, said pharmaceutical composition (B) may optionally be free of alpha-thrombin.

The pharmaceutical composition (B) of the present invention may further comprise at least one substance selected in the group consisting of growth factors, cytokines, hormones and combinations thereof. Said at least one substance may be selected in the group consisting of:
Bone morphogenetic proteins (BMP) such as BMP-1, BMP-2, BMP-5, BMP-6;
Epidermal growth factor (EGF);
Erythropoietin (EPO);
Fibroplast growth factors (FGF) such as FGF-1, FGF-4, FGF-5, FGF-12, FGF-13, FGF-15, FGF-20;
Granulocyte-colony stimulating factor (G-CSF);
Granulocyte-macrophage colony stimulating factor (GM-CSF);
Growth differentiation factor-9 (GDF-9);
Hepatocyte growth factor (HGF);
Insuline-like growth factor (IGF) such as IGF-2;
Myostatin (GDF-8);
Neurotrophins such as NT-3, NT-4, NT-1 and Nerve growth factor (NGF);
Platelet-derived growth factor (PDGF) such as PDGF-beta, PDGF-AA, PDGF-BB;
Thrombopoietin (TPO);
TGF—(Transforming growth factor alpha)
Transforming growth factors β, (TGF-β) such as TGF-β1, TGF-β2, TGF-β3;
VEGF (Vascular endothelial growth factor) such as VEGF-A, VEGF-C;
TNF-α, Leukemia inhibitory factor (LIF), interleukin 6 (IL-6), retinoic acid, C SDF-1 (stromal cell-derived factor-1), BDNF (brain-derived neurotrophic factor), Periostin, Angiotensin II, Flt3 Ligand, Glial-Derived Neurotrophic Factor, Heparin, Insulin-Like Growth Factor Binding Protein-3, Insulin-Like Growth Factor Binding Protein-5, Interleukin-3, Interleukin-8, Midkine, Progesterone, Putrescine, Stem Cell Factor, TGF-alpha, Wntl, Wnt3a, Wnt5a, caspase-4, chemokine ligand 1, chemokine ligand 2, chemokine ligand 5, chemokine ligand 7, chemokine ligand 11, chemokine ligand 20, haptoglobin, lectin, cholesterol 25-hydroxylase, syntaxin-8, syntaxin-11, ceruloplasmin, complement component 1, complement component 3, integrin alpha 6, lysosomal acid lipase 1, ν-2 microglobulin, ubiquitin, macrophage migration inhibitory factor, cofilin, cyclophillin A, FKBP12, NDPK, profilin 1, cystatin C, calcyclin, stanniocalcin-1, PGE-2, mpCCL2, IDO, iNOS, HLA-G5, M-CSF, angiopoietin, PIGF, MCP-1, extracellular matrix
molecules, CCL2 (MCP-1), CCL3 (MIP-1a), CCL4 (MIP-1β), CCL5 (RANTES), CCL7 (MCP-3), CCL20 (MIP-3a), CCL26 (eotaxin-3), CX3CL1 (fractalkine), CXCL5 (ENA-78), CXCL11 (i-TAC), CXCL1 (GROα), CXCL2 (GROβ), CXCL8 (IL-8), CCL10 (IP-10) and combinations thereof.

The stem cells to be stimulated may be resident cardiac stem cells (CSCs) or circulating stem cells or injected stem cells.

Said pharmaceutical composition may comprise primary particles. Said primary particles may be selected from the group consisting of alginates, chitosan, dextran, cellulose, liposome, or microspheres or nanospheres of polyesters such as PLGA, polycaprolactone or copolyesters. Preferably, said primary particles may encapsulate said at least two stem-cells-stimulating agents of said pharmaceutical composition (B). Hence, said primary particles may encapsulate the stem cells-stimulating agents comprised in the pharmaceutical composition (B). The term “primary” means that the pharmaceutical composition may be encapsulated in a first type of particles as defined above.

Preferably, said pharmaceutical composition (B) may be combined with a composition (A) comprising at least one pharmaceutically active substance to form a pharmaceutical cocktail. In one embodiment, said at least one pharmaceutically active substance may be selected in the group consisting of insulin-like growth factor 1 (IGF-1), hepatocyte growth factor (HGF) and/or variants thereof such as NK1, 1K1, 1K2, HP11, or HP21, and combinations thereof. In another embodiment, said composition (A) may further comprise SCF-1. Said composition (A) may further comprise secondary particles selected from the group consisting of alginates, chitosan, dextran, cellulose, liposomes, or microspheres or nanospheres of polyesters such as PLGA, polycaprolactone or copolyesters. Said secondary particles may encapsulate said at least one pharmaceutically active substance. The term “secondary” means that the composition (A) may be encapsulated in a second type of particles as defined above. In addition, said secondary particles may be configured to allow a delivery of the substances encapsulated therein before the delivery of the substance encapsulated in primary particles.

Said pharmaceutical cocktail may comprise a sample of said composition (B) and a sample of said composition (A). Alternatively, both compositions (A) and (B) may be mixed together in a single sample. When mixed together, compositions (A) and (B) may, however, be administrated to or delivered in the area, or surrounding the area, to be treated separately.

Said pharmaceutical composition of the present invention or said pharmaceutical cocktail may be used as medicine. Alternatively, said pharmaceutical composition of the present invention or said pharmaceutical cocktail may be used for the regeneration of cardiac tissue. Alternatively, said pharmaceutical composition of the present invention or said pharmaceutical cocktail may be used for the treatment of heart disease, including heart failure, heart ischemia or myocardial infarction.

In another aspect of the present invention, a process for acting in vivo or ex vivo on CSCs of human or animals is provided. Said process comprises the step of administrating said pharmaceutical composition (B) or said pharmaceutical cocktail of the present invention to said humans or animals.

The administration of the pharmaceutical composition (B) may follow a preliminary administration of a composition (A) comprising at least one pharmaceutically active substance.

The administration may be performed by sequential injection of composition A, B or of the cocktail.

Moreover, the duration between two successive administrations of said pharmaceutical composition or pharmaceutical cocktail of the present invention may be from one hour to 180 days. Each administration may be repeated. Alternatively, each or some administrations of said composition (A) may be optional.

Hence, said pharmaceutical composition (B) or pharmaceutical cocktail may be administrated parenterally. Moreover, said pharmaceutical composition (B) or pharmaceutical cocktail may be administrated into the circulatory system of a human or animal. Said pharmaceutical composition (B) or pharmaceutical cocktail may be administrated into veins and/or arteries.

The pharmaceutical composition (B) or pharmaceutical cocktail of the present invention may be administrated to a cardiac tissue. In the preferred embodiment, the administration may be intracoronary for said pharmaceutical composition (B) and intravenous for said preliminary administration of the composition (A).

TGFβ as used herein refers to TGFβ-1, TGFβ-2 or TGFβ-3 and can be any polypeptide having TGFβ activity, such as human TGFβ. For example, TGFβ can be recombinant TGFβ. In one embodiment, TGFβ can be TGFβ-1. Any appropriate concentration of TGFβ can be used. For example, between 0.1 and 100 ng of TGF-β per ml (e.g., about 33 ng of TGFβ1 per ml) can be used.

BMP can be any polypeptide having BMP activity, such as human BMP. For example, BMP can be recombinant BMP. In one embodiment, BMP can be BMP4. Any concentration of BMP can be used. For example, between 0.1 and 200 ng of BMP per ml (e.g., about 65 ng of BMP4 per ml) can be used.

FGF-2 can be any polypeptide having FGF-2 activity, such as human FGF-2. For example, FGF-2 can be recombinant FGF-2. Any concentration of FGF-2 can be used. For example, between 0.1 and 200 ng of FGF-2 per ml (e.g., about 65 ng of FGF-2 per ml) can be used.

IGF-1 can be any polypeptide having IGF-1 activity, such as human IGF-1. For example, IGF-1 can be recombinant IGF-1. Any concentration of IGF-1 can be used. For example, between 1 and 1000 ng of IGF-1 per ml (e.g., about 650 ng of IGF-1 per ml) can be used.

Activin-A can be any polypeptide having Activin-A activity, such as human Activin-A. For example, Activin-A can be recombinant Activin-A. Any concentration of Activin-A can be used. For example, between 0.1 and 500 ng of Activin-A per ml (e.g., about 130 ng of Activin-A per ml) can be used.

α-Thrombin can be any polypeptide having α-thrombin activity, such as human α-thrombin. For example, α-thrombin can be recombinant α-thrombin or synthetic α-thrombin. Any concentration of α-thrombin can be used. For example, between 0.05 and 100 units of α-thrombin per ml can be used.

Cardiotrophin can be any polypeptide having Cardiotrophin activity, such as human Cardiotrophin-1. For example, Cardiotrophin can be recombinant Cardiotrophin. Any concentration of Cardiotrophin can be used. For example, between 0.05 and 100 ng of Cardiotrophin per ml (e.g., about 13 ng of Cardiotrophin-1 per ml) can be used.

IL-6 can be any polypeptide having IL-6 activity, such as human IL-6. For example, IL-6 can be recombinant IL-6. Any concentration of IL-6 can be used. For example, between 10 and 400 ng of IL-6 per ml can be used.

Any concentration of Cardiogenol C or a pharmaceutically acceptable salt thereof (e.g., Cardiogenol C hydrochloride) can be used. For example, between 1 and 1000 ng of Cardiogenol C per ml (e.g., about 350 ng per ml of Cardiogenol C) can be used.

Retinoic acid can be any molecule having retinoic acid activity, such as synthetic retinoic acid, natural retinoic acid, a vitamin A metabolite, a natural derivative of vitamin A, or a synthetic derivative of vitamin A. Any concentration of retinoic acid can be used. For example, between 1×10⁻⁷ and 4×10⁻⁶ μM of retinoic acid can be used.

In some cases, serum-containing or serum-free media supplemented with TGFβ-1 (e.g., 2.5 ng/ml), BMP4 (e.g., 5 ng/ml), FGF-2 (e.g., 5 ng/ml), IGF-1 (e.g., 50 ng/ml), Activin-A (e.g., 10 ng/ml), Cardiotrophin (e.g., 1 ng/ml), α-thrombin (e.g., 1 Unit/ml), and Cardiogenol C (e.g., 100 nM) can be used. In some cases, the media (e.g., serum-containing or serum-free media) can contain platelet lysate (e.g., a human platelet lysate).

In some cases, the composition used to stimulate CSCs may contain additional optional factors such as TNF-α, LIF, and VEGF-A.

TNF-αcan be any polypeptide having TNF-αactivity, such as human TNF-α. For example, TNF-αcan be recombinant TNF-α. Any concentration of TNF-α can be used. For example, between 0.5 and 100 ng of TNF-α per ml can be used.

LIF can be any polypeptide having LIF activity, such as human LIF. For example, LIF can be recombinant LIF. Any concentration of LIF can be used. For example, between 0.25 and 200 ng of LIF per ml can be used.

VEGF-A can be any polypeptide having VEGF-A activity, such as human VEGF-A. For example, VEGF-A can be recombinant VEGF-A. Any concentration of VEGF-A can be used. For example, between 0.5 and 400 ng of VEGF-A per ml can be used.

A composition provided herein can be prepared using any appropriate method. For example, a composition provided herein can be prepared using commercially available stimulating agents. In some cases, a composition provided herein can be prepared to contain cells lysates (e.g., a platelet lysate) or conditioned media from cells such as cardiomyocyte cells or TNF-α-stimulated endodermal cells. For example, a composition provided herein can be prepared using a platelet lysate supplemented with commercially available factors. In some cases, a composition provided herein can be prepared using factors isolated from conditioned medium. In some cases, the factors can be dissolved in media such as cell culture media that does or does not contain serum.

### EXAMPLES

Tests are performed in acutely infarcted pigs. The following protocol is established. The infarct is performed at T0 by 90 minutes left anterior descending (LAD) occlusion followed by a 30 minutes reperfusion. At the end of the reperfusion (T1), a primary composition is parenterally administrated to the animals by intracoronary delivery distal to the occlusion site. A BrdU loaded osmotic pump is also subcutaneously implanted at T1. Fourteen days later (T2), a secondary composition is parenterally administrated to the animals. The administration of both compositions can be achieved with different methods of administration such as intravenous injection, intramuscular injection or intracoronary injection. Finally, at 42 days (T3), the euthanasia of the pigs is performed.

The concentration of constituents are mentioned in brackets. Two compositions, alone or in combination, are tested:
Composition A consists of IGF-1 (8 μg in 15 ml of Phosphate Buffer Solution (PBS)) and HGF (2 μg in 15 ml of PBS) and
Composition B consists of TGFβ-1 (0.5 μg in 15 ml of PBS), BMP4 (1 μg in 15 ml of PBS), FGF-2 (1 μg in 15 ml of PBS), IGF-1 (10 μg in 15 ml of PBS), Activin-A (2 μg in 15 ml of PBS), Cardiotrophin 1 (0.2 μg in 15 ml of PBS) and Cardiogenol C (5.2 μg in 15 ml of PBS).
   Both compositions are in a pharmaceutically acceptable excipient. The pharmaceutically acceptable excipient may be phosphate buffered solution (PBS), Hartmann's solution, Ringer's lactate, physiological NaCI (0.9% NaCI) supplemented or not with albumin or with any suitable protein stabilizer composition.

Five treatment groups of 5 animals each are evaluated.
Treatment group 1 is a control group and only receives saline solution at T1 and T2.
Treatment group 2 receives a solution containing the composition A at T1 and a saline solution at T2, noted as mix A.
Treatment group 3 receives a solution containing the composition A at T1 and a solution containing the composition B at T2, noted mix A+B.
Treatment group 4 receives a solution containing the composition B at T1 and T2, noted mix B+B.
Treatment group 5 receives a solution containing the composition B at T1 and a saline solution at T2, noted mix B.

The protocol is summarized in table 1.

TABLE 1

| | Treatment | Treatment | Treatment | Treatment | Treatment |
|---|---|---|---|---|---|
| | group 1 | group 2 | group 3 | group 4 | group 5 |
| T0: infarct | | | | | |
| T1: 30 min | Saline | A | A | B | B |
| T2: 2 weeks | Saline | Saline | B | B | Saline |
| T3: 6 weeks | Euthanasia | Euthanasia | Euthanasia | Euthanasia | Euthanasia |

Blood analyses were performed at different intervals. Blood samples are collected from 2 pigs per treatment group in coronary sinus via jugular vein and venous blood via ear vein. After the primary administration, samples are collected at T1+5 min; T1+1 h and T1+6 h. After the secondary administration, samples are collected at T2+5 min; T2+1 h, T2+6 h and T2+24 h. ELISA immunoassays are performed with samples for the quantification of IGF-1 and cardiotrophin 1 concentration.

Magnetic resonance imaging (MRI) is also performed on all animals at T1+3 days; T2 and T3 to study the scar area, the global left ventricular function, the regional function (wall motion and thickening) and regional perfusion of the ventricular. MRI allows to detect and confirm the presence of new vessels, tissue or cells improving ventricular function.

Histopathology is also performed to determine the scar area, the identification and quantification of c-kit positive cardiac stem cells. Histopathology also provides data on distribution, size and density of new vessels and cardiomyocytes. Histopathology allows documenting the repair process at the tissue and cellular level.

Critical variables have been considered in the analysis of cardiac repair: (1) amount of reconstituted tissue or myocardium mass and coronary vasculature; (2) number and size of restored myocytes and vessels; (3) integration of newly formed myocytes and vessels with the surrounding myocardium; and/or (4) origin of the regenerated myocardial structures.

Infarct Result

Images from MRI imaging were used to evaluate infarct size, infarct weight and the infarct area. Results are listed in Table 2 below.

TABLE 2

| | Infarct area | Infarct weight | Infarct Volume |
|---|---|---|---|
| | (%) | (g) | (cm³) |
| Group 1 | 19.8 | 15.6 | 22.0 |
| (Control) | | | |
| Group 2 | 18.7 | 15.8 | 20.7 |
| (Mix A) | | | |
| Group 5 | 13.7 | 13.2 | 15.6 |
| (Mix B) | | | |
| Group 4 | 18.8 | 22.2 | 25.2 |
| (Mix B + B) | | | |
| Group 3 | 9.6 | 10.3 | 10.3 |
| (Mix A + B) | | | |

Experiments demonstrate that the infarct area was about 19.8% for the control group and about 19% for the group 2 and 4 wherein mix A and mix B+B respectively was used. Surprisingly, using the composition (B) according to the present invention, the infarct area was limited to 13.7% for the group 5 (mix B). Hence, the composition (B) according to the present invention was very efficient to treat infarct, such as myocardial infarction, compared to the other composition.

In addition, it was also surprisingly observed that when the injection of mix B followed the preliminary injection of mix A, the infarct area was further limited to the value of about 9.6%. This is a result which would not be expected based on the results observed for the other groups. Indeed, mix A used for the group 2 was almost inefficient alone. A synergistic effect was observed by using a pharmaceutical cocktail according to the present invention. This result was confirmed with histopathology and immunohistochemistry testing.

Histopathology

Results were compiled separately for sections taken in the border zone or within the central areas of the infarct. Results for all the groups are listed in Table 3 below. Data showed in Table 3 are mean from heart slices analyzed for an animal of each group.

TABLE 3

| | Border zone | | Infarct center | |
|---|---|---|---|---|
| | Infarct/ | Transmurality | Infarct/ | Transmurality |
| | scar (%) | (%) | scar (%) | (%) |
| Group 1 | 33.0 | 4.0 | 73.3 | 13.3 |
| (Control) | | | | |
| Group 2 | 36.0 | 6.0 | 56.7 | 6.7 |
| (Mix A) | | | | |
| Group 5 | 26.0 | 6.0 | 30.0 | 0.0 |
| (Mix B) | | | | |
| Group 4 | 31.4 | 12.9 | 70.0 | 10.0 |
| (Mix B + B) | | | | |
| Group 3 | 20.0 | 0.0 | 25.0 | 0.0 |
| (Mix A + B) | | | | |

The ratio between the infarct and scar size represents the infarct size while the transmurality is a parameter establishing whether the infarct is strongly localized at the external surface of the myocardium or it extends throughout the internal surface of the myocardium. The higher is the transmurality value, the larger is the infarct.

With regard to the border zone of the infarct, Table 3 shows that mix A, mix B+B or control mix had almost no impact on the infarct size. In those cases, the ratio between the infarct and scar size ranged from 31.4% to 36.0%. On the contrary, when the composition according to the present invention is used (i.e. mix B), the ratio between infarct and scar size was surprisingly decreased to 26%. This value can be further decreased up to 20% when the pharmaceutical cocktail according to the present invention (i.e. mix A+B) was used. This experiment demonstrates that the present pharmaceutical composition and pharmaceutical cocktail are effective to treat heart disease. This was also confirmed when experiments were performed in the infarct zone.

Furthermore, it was surprisingly demonstrated that transmurality is reduced with mix B alone or mix A+B. This means that the composition (B) of the present invention alone or when combined with composition (A) allows limiting the expansion of the infarct to the external surface of the myocardium. This is another evidence that the pharmaceutical composition and the pharmaceutical cocktail according to the present invention are powerful compositions to treat heart diseases or troubles.

Hence, it is clear from the above-described experiments that both pharmaceutical composition and pharmaceutical cocktail according to the present invention are suitable for the treatment of heart failure secondary to myocardial ischemia, ischemia or myocardial infarction.

Immunohistochemistry

Tests were performed to evaluate, within the infarct sections, the microvessel density (vWF-positive vessels/mm²), BrdU positive cells and c-kit positive cells. The quantification of microvessel density using von Willebrand factor (vWF) allows determining the amount of new blood vessels created in the infarct zone. BrdU positive cells tests represent the proliferation of cells, including cardiac cells. C-kit positive cells tests show the amount of CSCs within the selected infarct sections. Results are listed in Table 4. These testing were only performed for group 1 (Control group), group 3 (Mix A+B) and group 5 (Mix B).

TABLE 4

| | Group 1 | Group 3 | Group 5 |
|---|---|---|---|
| | (Control) | (Mix A + B) | (Mix B) |
| Microvessel Density | 27.9 | 34.3 | 34.3 |
| (vWF-positive vesels/mm²) | | | |
| BrdU positive cells (%) | 22.1 | 52.7 | 36.0 |
| c-kit positive cells (%) | 1.9 | 1.2 | 1.8 |

Results show that when compositions (A) and (B) in combination or composition (B) alone, according to the invention, are injected in the heart, they have great impact on cardiac stem cells stimulation or cardiac cells proliferation. Indeed, microvessel density is enhanced and new blood vessels were created upon stimulation with the present composition or present cocktail. Results obtained with groups 3 or 5 reached 34.2 and 34.3 respectively, compared to 27.9 for the control group. This is confirmed with BrdU positive cells test which shows that cells proliferation was enhanced with the composition of the present invention and that strong cellular activity was observed. When Mix B was injected, 36.0% of BrdU positive cells were observed compared to only 22.1% for the control group. This clearly highlights that the pharmaceutical composition according to the present invention promotes cellular proliferation and thus the formation of new myocytes and vessels with the surrounding myocardium. This can be further enhanced when the pharmaceutical cocktail according to the present invention was used. A value of 52.7% was reached with such cocktail. Hence, both pharmaceutical composition and pharmaceutical cocktail according to the present invention are suitable for improving heart tissue regeneration.

The ability of the pharmaceutical cocktail to induce and to promote the CSCs activation and proliferation was confirmed with c-kit positive cells test. C-kit positive cells test allows demonstrating that resident CSCs are consumed since their amount has significantly decreased when mix A+B was used compared to the control group. Hence, the regenerated myocardial structures are originated from resident cardiac stem cells. The present composition and/or cocktail are effective for in vivo stimulation of resident cardiac stem cells.

The terms and descriptions used herein are set forth by way of illustration only and are not meant as limitations. Those skilled in the art will recognize that many variations are possible within the spirit and scope of the invention as defined in the following claims, and their equivalents, in which all terms are to be understood in their broadest possible sense unless otherwise indicated. As a consequence, all modifications and alterations will occur to others upon reading and understanding the previous description of the invention. In particular, dimensions, materials, and other parameters, given in the above description may vary depending on the needs of the application.

1. A pharmaceutical composition (B) for the stimulation of stem cells, comprising at least two stem cells-stimulating-agents and at least one pharmaceutically acceptable excipient, wherein the at least two stem-cells-stimulating-agents are selected from the group consisting of TGFβ-1, BMP-4, FGF-2, IGF-1, Activin-A, Cardiotrophin 1, Cardiogenol C and mixtures thereof.

2. (canceled)

3. The pharmaceutical composition according to claim 1, wherein the stem-cells-stimulating-agents consisting essentially of TGFβ-1, BMP-4, FGF-2, IGF-1, Activin-A, Cardiotrophin 1, or Cardiogenol C.

4. (canceled)

5. The pharmaceutical composition according to claim 1, which further comprises at least one substance selected in the group consisting of growth factors, cytokines, hormones and a combination thereof.

6. A pharmaceutical composition according to claim 5, wherein the at least one substance is selected in the group consisting of
Bone morphogenetic proteins (BMP) such as BMP-1, BMP-2, BMP-5, BMP-6;
Epidermal growth factor (EGF);
Erythropoietin (EPO);
Fibroplast growth factors (FGF) such as FGF-1, FGF-4, FGF-5, FGF-12, FGF-13, FGF-15, FGF-20;
Granulocyte-colony stimulating factor (G-CSF);
Granulocyte-macrophage colony stimulating factor (GM-CSF);
Growth differentiation factor-9 (GDF-9);
Hepatocyte growth factor (HGF);
Insuline-like growth factor (IGF) such as IGF-2;
Myostatin (GDF-8);
Neurotrophins such as NT-3, NT-4, NT-1 and Nerve growth factor (NGF);
Platelet-derived growth factor (PDGF) such as PDGF-beta, PDGF-AA, PDGF-BB;
Thrombopoietin (TPO);
Transforming growth factor alpha (TGF-α)
Transforming growth factors β, (TGF-β) such as TGF-β1, TGF-β2, TGF-β3;
VEGF (Vascular endothelial growth factor) such as VEGF-A, VEGF-C;
TNF-α, Leukemia inhibitory factor (LIF), interleukin 6 (IL-6), retinoic acid, C SDF-1 (stromal cell-derived factor-1), BDNF (brain-derived neurotrophic factor), Periostin, Angiotensin II, Flt3 Ligand, Glial-Derived Neurotrophic Factor, Insulin-Like Growth Factor Binding Protein-3, Insulin-Like Growth Factor Binding Protein-5, Interleukin-3, lnterleukin-8, Midkine, Progesterone, Putrescine, Stem Cell Factor, TGF-alpha, Wntl, Wnt3a, Wnt5a, caspase-4, chemokine ligand 1, chemokine ligand 2, chemokine ligand 5, chemokine ligand 7, chemokine ligand 11, chemokine ligand 20, haptoglobin, lectin, cholesterol 25-hydroxylase, syntaxin-8, syntaxin-11, ceruloplasmin, complement component 1, complement component 3, integrin alpha 6, lysosomal acid lipase 1,ν-2 microglobulin, ubiquitin, macrophage migration inhibitory factor, cofilin, cyclophillin A, FKBP12, NDPK, profilin 1, cystatin C, calcyclin, stanniocalcin-1, PGE-2, mpCCL2, IDO, iNOS, HLA-G5, M-CSF, angiopoietin, PIGF, MCP-1, extracellular matrix molecules, CCL2 (MCP-1), CCL3 (MIP-1α), CCL4 (MIP-1β), CCL5 (RANTES), CCL7 (MCP-3), CCL20 (MIP-3α), CCL26 (eotaxin-3), CX3CL1 (fractal kine), CXCL5 (ENA-78), CXCL11 (i-TAC), CXCL1 (GROα), CXCL2 (GROβ), CXCL8 (IL-8), CCL10 (IP-10) and combinations thereof.

7. The pharmaceutical composition according to claim 1, wherein stem cells to be stimulated are resident cardiac stem cells or circulating stem cells or injected stem cells.

8. The pharmaceutical composition according to claim 1, which further comprises primary particles.

9. The pharmaceutical composition according to claim 8, wherein the primary particles are selected from the group consisting of alginates, chitosan, dextran, cellulose, liposome, microspheres of polyesters, and nanospheres of polyesters.

10. The pharmaceutical composition according to claim 8, wherein the primary particles encapsulate the stem cells-stimulating agents of said pharmaceutical composition.

11. A pharmaceutical preparation comprising the pharmaceutical composition (B) according to claim 1, and a composition (A) comprising at least one pharmaceutically active substance selected from the group consisting of insulin-like growth factor 1 (IGF-1), hepatocyte growth factor (HGF), and an HGF variant.

12. (canceled)

13. The pharmaceutical preparation according to claim 11, wherein said composition A further comprises SCF-1.

14. The pharmaceutical preparation according to claim 11, wherein said composition (A) further comprises secondary particles selected from the group consisting of alginates, chitosan, dextran, cellulose, liposome, microspheres of polyesters, and nanospheres of polyesters.

15. The pharmaceutical preparation according to claim 14, wherein secondary particles encapsulate said at least one pharmaceutically active substance.

16. The pharmaceutical preparation according to claim 14, wherein said secondary particles are configured to allow a delivery of the substance encapsulated therein before the delivery of the substance encapsulated in primary particles.

17-32. (canceled)

33. The pharmaceutical composition of claim 1, wherein the composition further comprises a thrombin inhibitor.

34. The pharmaceutical composition of claim 9, wherein said polyesters are selected from the group consisting of PLGA, polycaprolactone and copolyesters.

35. The pharmaceutical composition of claim 14, wherein said polyesters are selected from the group consisting of PLGA, polycaprolactone and copolyesters.

36. The pharmaceutical preparation according to claim 11, wherein the HGF variant is NK1, 1K1, 1K2, HP11, HP21, or a combination thereof.

37. A method for treating heart disease-in a mammal in need thereof, comprising administering the pharmaceutical composition (B) of claim 1 to said mammal.

38. The method of claim 37, further comprising the administration of a composition (A), wherein the composition (A) comprises at least one pharmaceutically active substance selected from the group consisting of: insulin-like growth factor 1 (IGF-1); hepatocyte growth factor (HGF), and an HGF variant.

39. The method of claim 38, wherein the pharmaceutical composition (B) and the composition (A) are administered simultaneously.

40. The method of claim 38, wherein the administration of the pharmaceutical composition (B) follows the administration of the composition (A).

41. The method of claim 40, wherein the duration between the administration of the pharmaceutical composition (B) and the administration of the composition (A) is up to approximately two weeks.

42. The method of claim 37, wherein the administration of the pharmaceutical composition (B) is repeated.

43. The method of claim 38, wherein at least one of the administrations is repeated.

44. The method of claim 42, wherein the duration between two successive administrations of the pharmaceutical composition (B) is from 1 hour to 180 days.

45. The method of claim 38, wherein each administration is in vivo.

46. The method of claim 38, wherein each administration is performed by injection.

47. The method of claim 46, wherein the injection is a sequential injection.

48. The method of claim 38, wherein each administration is administered parenterally.

49. The method of claim 38, wherein each administration is administered to cardiac tissue.

50. The method of claim 38, wherein each administration is administered into the circulatory system of a mammal.

51. The method of claim 38, wherein each administration is administered into veins and/or arteries.

52. The method of claim 38, wherein the administration of pharmaceutical composition (B) is intracoronary, and the administration of composition (A) is intravenous.
